(19) United States
(12) Patent Application Publication
(10) Pub. No.: US 2009/0124929 A1
(43) Pub. Date: May 14, 2009

(54) Feminine urine sample collection device with screw-on led and detachable support and handle

(76) Inventors: Maria Rossi-Pipitone, Hudson, OH (US); Assunta Rossi, Westlake, OH (US)

Correspondence Address: ASSUNTA ROSSI, 1333 COBBLESTONE CHASE, WESTLAKE, OH, 44145, US

(21) Appl. No.: 12/218,090

(22) Filed: Jul. 12, 2008

Related U.S. Application Data
(60) Provisional application No. 60/929,764, filed on Jul. 12, 2007.

(51) Int. Cl. A61F 5/455

(52) U.S. Cl. 600/574

(57) ABSTRACT

The invention provides an improved urine sample gathering device specifically for women. The device permits a woman to position the device with ease, control and comfort, ensures that the urine will sanitarily and effectively collect in the sample bottle and prevents any actual contact with the urine. The device is comprised of 4 separate, detachable and disposable parts that include the following: (1) a curved, oval basin, cup or apparatus shaped to fit comfortably and securely around the female genitalia that screws over a container or bottle and is detachable and disposable; (2) a container or bottle in which the urine will collect and can be stored; (3) an underlying lid which unscrews from the bottom of the container or bottle to secure the urine sample once it is collected; (4) an outer support apparatus or handle that is detachable and disposable with a curved hook-like end to permit handling or storage of the entire urine sample device.

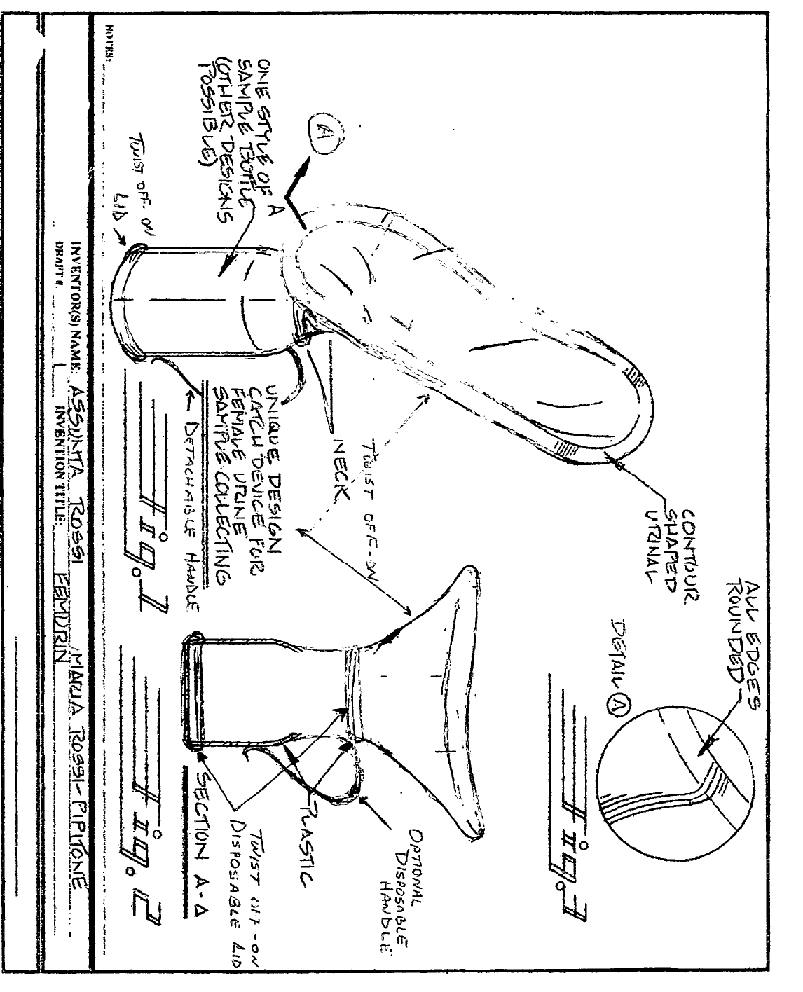

### FIELD OF THE INVENTION

The invention relates to a feminine urine sample collection device with screw-on lid and detachable support and handle.

### BACKGROUND OF THE INVENTION

This invention relates to a feminine urine sample collection device with a screw-on detachable support, lid and handle. It serves mainly to facilitate and effectively gather female urine samples. Urine sample devices (containers) that are used by all medical and health facilities and professionals are basically small tubes, bottles or cups that have a snap-on or screw-on cap or lid. This invention comprises of three adjustable, disposable and effective pieces. This device allows the female user to adjust and secure the device through the use of the handle and the curved holder. The device also prevents spills or leakage during the taking of any urine sample. The curved holder fits comfortably and securely around the vaginal area. The handle allows the female user to comfortably and sanitarily position the device to ensure a proper sample.

### BRIEF DESCRIPTION OF THE DRAWING

The device features a curved-oval shaped opening with rounded edges that covers and cups around the private area and is screwed on top of a bottle or cup for the urine collection and is disposable. The bottle or cup can be any shape or size with a detachable, disposable handle and a twist on/off lid that fits on top of the bottle or cup to secure the specimen when collected. The drawing shows a three-dimensional view and a side view of the device.

1. a detachable and disposable, curved, oval shaped basin or cup holder that screws and sits on top of the urine sample bottle or container.

2. the holder defined in claim #1 wherein it is detachable from the urine sample bottle or container.

3. the holder defined in claim #1 wherein it is disposable from the urine sample bottle or container

4. the holder defined in claim #1 wherein it is so designed as to fit securely and comfortably around the female vaginal frame to prevent any leakage.

5. the holder defined in claim #1 screws on and fits on top of a urine sample bottle or container that has a detachable and disposable handle.

6. the handle defined in claim #5 is either straight or hooked around the urine sample bottle or container to allow for and easy and proper grip and placement of the entire device

7. the handle defined in claim #5 will snap or break off of the urine sample bottle or container and can be disposed of

8. the holder defined in claim #1 and the handle defined in claim #5 attach to the urine sample bottle or container that is also disposable

9. the urine sample bottle or container defined in claim #8 can be sized to hold the medically or scientifically required amount of urine necessary for sampling and analysis

10. the urine sample bottle or container defined in claim #8 has a the bottom of the bottle or container a detachable, screw-on lid or cover that can be easily unscrewed from the bottom and screwed on top when the holder defined in claim #1 is unscrewed or removed

11. the lid defined in claim #10 can be easily unscrewed from the bottom of the urine sample bottle or container and screwed on top of the urine sample bottle or container so as to secure the urine sample in the bottle or container.
